(19) United States
(12) Patent Application Publication
(10) Pub. No.: US 2026/0144504 A1
(43) Pub. Date: May 28, 2026

(54) MEDICAL IMAGE CAPTURING SUPPORT APPARATUS, OPERATION METHOD OF MEDICAL IMAGE CAPTURING SUPPORT APPARATUS, PROGRAM, AND MEDICAL IMAGE CAPTURING SYSTEM

(71) Applicant: FUJIFILM Corporation, Tokyo (JP)

(72) Inventor: Takeshi HIRAYU, Tokyo (JP)

(73) Assignee: FUJIFILM Corporation, Tokyo (JP)

(21) Appl. No.: 19/367,858

(22) Filed: Oct. 24, 2025

(30) Foreign Application Priority Data
Nov. 28, 2024 (JP) 2024-207561

(51) Int. Cl. A61B 6/04; A61B 6/00; A61B 6/03

(52) U.S. Cl. CPC A61B 6/0407; A61B 6/03; A61B 6/0492; A61B 6/4411; A61B 6/5258; A61B 6/547

(57) ABSTRACT

Provided are a medical image capturing support apparatus, an operation method of a medical image capturing support apparatus, a program, and a medical image capturing system that can determine whether or not there is misalignment of a mounting state of an imaging apparatus that acquires a captured image of a subject. A medical image capturing support apparatus acquires a first captured image of a subject placed on a bed, the first captured image being acquired by imaging the subject using an imaging apparatus, detects an adjustment reference defined on the bed from the first captured image, derives a difference between a captured image reference set for the first captured image and the adjustment reference, and determines misalignment of a mounting state of the imaging apparatus based on the difference.

### CROSS-REFERENCE TO RELATED APPLICATION

The present application claims priority under 35 U.S.C § 119(a) to Japanese Patent Application No. 2024-207561 filed on Nov. 28, 2024, which is hereby expressly incorporated by reference, in its entirety, into the present application.

### BACKGROUND OF THE INVENTION

### 1. Field of the Invention

The present disclosure relates to a medical image capturing support apparatus, an operation method of a medical image capturing support apparatus, a program, and a medical image capturing system.

### 2. Description of the Related Art

In a medical image capturing apparatus such as an MRI apparatus and an X-ray CT apparatus, an operator sets imaging conditions such as a body position of a patient in advance, and then places the patient on an examination bed in a shielded room and starts a scan, thereby starting an examination. The operator represents a person who operates the medical image capturing apparatus, such as a technician. The patient is a person to be examined, and can be referred to as a subject, an examinee, an examination subject, and the like.

It should be noted that MRI is an abbreviation for Magnetic Resonance Imaging, which is an English notation. CT is an abbreviation for Computed Tomography.

In the medical image capturing apparatus in which a camera is disposed on a ceiling or the like of the shielded room, the subject set on the bed is imaged using the camera, and movement of the bed can be controlled in capturing a medical image based on a captured image of the subject.

JP2016-538957A discloses an MRI apparatus that images a head of a patient using a camera mounted to a head coil and that monitors movement of the head of the patient. In the apparatus disclosed in JP2016-538957A, in cross-calibration between an MRI coordinate and an optical coordinate system applied to the camera, a difference between a current position and a reference position of the camera is derived.

JP2003-319930A discloses a positioning method that enables repeated positioning of a patient in an image diagnostic apparatus or a radiotherapy apparatus. In the method disclosed in JP2003-319930A, a positional shift of the patient is derived from a difference between an image of the patient at a first reference position and an image of the same patient captured using the same camera position, viewpoint, and camera axis in the same apparatus.

### SUMMARY OF THE INVENTION

However, due to occurrence of an accidental contact with the camera disposed on a ceiling or the like or due to vibration of the camera caused by an earthquake or the like, a mounting state of the camera adjusted in advance may be misaligned. The misalignment of the mounting state of the camera may cause a reduction in accuracy in movement control of the bed in which a captured image of the subject is used.

The apparatus disclosed in JP2016-538957A derives a difference between a current position and a reference position of the camera in cross-calibration between a coordinate system of an MRI scanner and a coordinate system of the camera mounted to the head coil or the like, but does not solve the above-mentioned problem caused by the misalignment of the mounting state of the camera mounted to the head coil or the like.

The method disclosed in JP2003-319930A involves reproducing a patient position applied to first imaging in subsequent imaging in a case of acquiring a medical image of the same patient using the same apparatus, and does not solve the above-mentioned problem caused by the misalignment of the mounting state of the camera.

The present disclosure has been made in view of such circumstances, and an object of the present disclosure is to provide a medical image capturing support apparatus, an operation method of a medical image capturing support apparatus, a program, and a medical image capturing system that can determine whether or not there is misalignment of a mounting state of an imaging apparatus that acquires a captured image of a subject.

According to a first aspect of the present disclosure, there is provided a medical image capturing support apparatus comprising: a processor; and a memory in which a program to be executed by the processor is stored, in which the processor is configured to acquire a first captured image of a subject placed on a bed, the first captured image being acquired by imaging the subject using an imaging apparatus, detect an adjustment reference defined on the bed from the first captured image, derive a difference between a captured image reference set for the first captured image and the adjustment reference, and determine misalignment of a mounting state of the imaging apparatus based on the difference.

With the medical image capturing support apparatus according to the first aspect, the misalignment of the mounting state of the imaging apparatus that acquires the captured image is determined based on the difference between the captured image reference set for the captured image of the subject and the adjustment reference detected from the captured image. As a result, it is possible to understand whether or not there is the misalignment of the mounting state of the imaging apparatus based on a determination result.

The difference between the captured image reference and the adjustment reference may be a difference between a position of the captured image reference and a position of the adjustment reference. The position of the captured image reference and the position of the adjustment reference may be represented by using coordinate values in the captured image.

The difference between the captured image reference and the adjustment reference may be a difference between an orientation of the captured image reference and an orientation of the adjustment reference. The orientation of the captured image reference and the orientation of the adjustment reference may be represented by using a rotation angle in a plane of the captured image.

According to a second aspect, in the medical image capturing support apparatus according to the first aspect, the processor may be configured to determine that the mounting state of the imaging apparatus is misaligned in a case where the difference exceeds a first range.

According to a third aspect, in the medical image capturing support apparatus according to the first or second aspect, the processor may be configured to output a control signal for correcting movement control of the bed based on the difference.

According to a fourth aspect, in the medical image capturing support apparatus according to any one of the first to third aspects, the processor may be configured to issue a warning in a case where the difference exceeds a second range.

According to a fifth aspect, in the medical image capturing support apparatus according to any one of the first to fourth aspects, the processor may be configured to detect a light localizer displayed on the bed as the adjustment reference.

In the fifth aspect, the processor may detect a position of the light localizer beam in the first captured image as a position of the adjustment reference.

According to a sixth aspect, in the medical image capturing support apparatus according to any one of the first to fourth aspects, the processor may be configured to detect an image marker represented on the bed as the adjustment reference.

According to a seventh aspect, in the medical image capturing support apparatus according to the sixth aspect, the processor may be configured to detect a position of the image marker in the first captured image as a position of the adjustment reference.

According to an eighth aspect, in the medical image capturing support apparatus according to the sixth aspect, the processor may be configured to acquire a shape of the image marker represented on the bed as the captured image reference, detect a shape of the image marker in the first captured image as the adjustment reference, and derive a difference between the shape of the image marker represented on the bed and the shape of the image marker in the first captured image.

According to a ninth aspect, in the medical image capturing support apparatus according to any one of the first to fourth aspects, the processor may be configured to detect a structure of the bed as the adjustment reference.

In the ninth aspect, the processor may detect a position of the structure of the bed as the position of the adjustment reference.

According to a tenth aspect, in the medical image capturing support apparatus according to any one of the first to ninth aspects, the processor may be configured to set the adjustment reference included in a second captured image acquired by using the imaging apparatus in a specified mounting state, as the captured image reference.

According to an eleventh aspect of the present disclosure, there is provided an operation method of a medical image capturing support apparatus, the operation method comprising causing a computer functioning as the medical image capturing support apparatus to execute: acquiring a first captured image of a subject placed on a bed, the first captured image being acquired by imaging the subject using an imaging apparatus; detecting an adjustment reference defined on the bed from the first captured image; deriving a difference between a captured image reference set for the first captured image and the adjustment reference; and determining misalignment of a mounting state of the imaging apparatus based on the difference.

According to the operation method of a medical image capturing support apparatus according to the eleventh aspect of the present disclosure, it is possible to obtain the same actions and effects as those of the medical image capturing support apparatus according to the first aspect. Configuration requirements of the medical image capturing support apparatus according to the second to tenth aspects can be applied as configuration requirements of the operation method of a medical image capturing support apparatus according to other aspects.

According to a twelfth aspect of the present disclosure, there is provided a program for causing a computer functioning as a medical image capturing support apparatus to realize: a function of acquiring a first captured image of a subject placed on a bed, the first captured image being acquired by imaging the subject using an imaging apparatus; a function of detecting an adjustment reference defined on the bed from the first captured image; a function of deriving a difference between a captured image reference set for the first captured image and the adjustment reference; and a function of determining misalignment of a mounting state of the imaging apparatus based on the difference.

With the program according to the twelfth aspect of the present disclosure, it is possible to obtain the same actions and effects as those of the medical image capturing support apparatus according to the first aspect. Configuration requirements of the medical image capturing support apparatus according to the second to tenth aspects can be applied as configuration requirements of a program according to other aspects.

According to a thirteenth aspect of the present disclosure, there is provided a medical image capturing system comprising: an imaging apparatus configured to image a subject and generate imaging data of the subject; an image reconstruction unit configured to generate a reconstructed image based on the imaging data; a processor; and a memory in which a program to be executed by the processor is stored, in which the processor is configured to acquire a first captured image of a subject placed on a bed, the first captured image being acquired by imaging the subject using an imaging apparatus, detect an adjustment reference defined on the bed from the first captured image, derive a difference between a captured image reference set for the first captured image and the adjustment reference, and determine misalignment of a mounting state of the imaging apparatus based on the difference.

With the medical image capturing system according to the thirteenth aspect of the present disclosure, it is possible to obtain the same actions and effects as those of the medical image capturing support apparatus according to the first aspect. Configuration requirements of the medical image capturing support apparatus according to the second to tenth aspects can be applied as configuration requirements of a medical image capturing system according to other aspects.

According to the present disclosure, the misalignment of the mounting state of the imaging apparatus that acquires the captured image is determined based on the difference between the captured image reference set for the captured image of the subject and the adjustment reference detected from the captured image. As a result, it is possible to understand whether or not there is the misalignment of the mounting state of the imaging apparatus based on a determination result.

### BRIEF DESCRIPTION OF THE DRAWINGS

FIG. 1 is a schematic configuration diagram of a medical image capturing system according to an embodiment.

FIG. 2 is a functional block diagram showing an electric configuration example of an automatic imaging position movement controller.

FIG. 3 is a block diagram showing a hardware configuration example of an electric configuration of an operation unit.

FIG. 4 is a flowchart showing a procedure of an automatic imaging position movement correction method.

FIG. 5 is a schematic diagram of a camera image.

FIG. 6 is a schematic diagram of a camera image in a case where a mounting state of a camera is misaligned in front, rear, left, and right directions.

FIG. 7 is a schematic diagram of a camera image in a case where the mounting state of the camera is rotationally misaligned.

FIG. 8 is a schematic diagram of a camera image showing a specific example of a camera image reference.

FIG. 9 is a schematic diagram showing a first specific example of an adjustment reference.

FIG. 10 is a schematic diagram showing a second specific example of the adjustment reference.

FIG. 11 is an explanatory diagram of a modification example of an image marker.

### DESCRIPTION OF THE PREFERRED EMBODIMENTS

Hereinafter, a preferred embodiment of the present invention will be described in detail with reference to the accompanying drawings. In the following description and the accompanying drawings, the same constituent elements are denoted by the same reference numerals, and the duplicated description thereof is omitted. In addition, in the following embodiment, in a case where a plurality of constituent elements are described and listed, it can be interpreted that at least one of the plurality of constituent elements is included.

### Configuration Example of Medical Image Capturing System

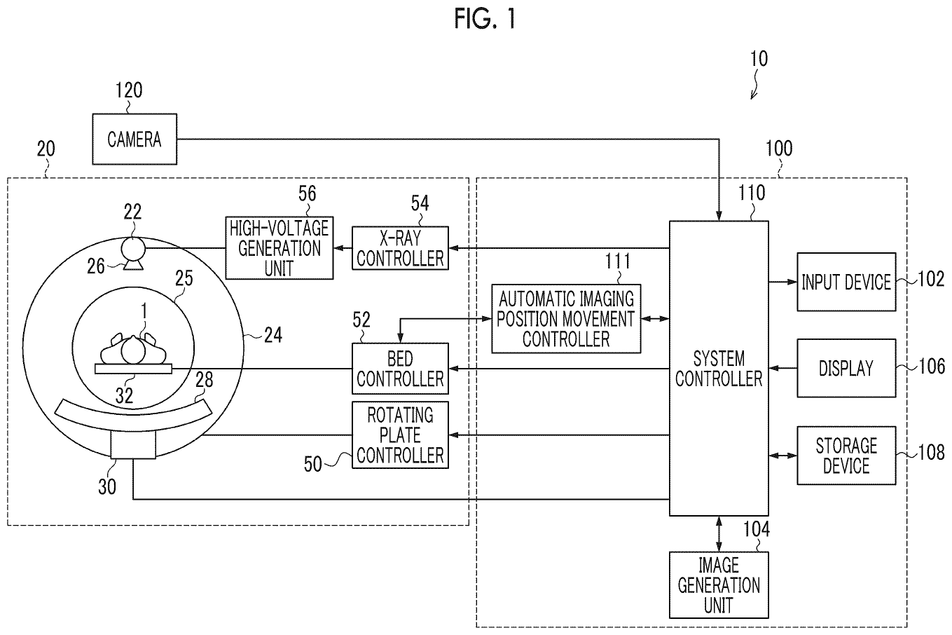

FIG. 1 is a schematic configuration diagram of a medical image capturing system according to an embodiment. A medical image capturing system 10 is a system that captures a medical image used for diagnosing a subject 1 by detecting X-rays transmitted through the subject 1 and a nuclear magnetic resonance signal generated from the subject 1.

Hereinafter, as an example of the medical image capturing system 10, an X-ray CT system that acquires an X-ray projection image of the subject 1 at various projection angles and that acquires a tomographic image of the subject 1 will be illustrated.

The medical image capturing system 10 shown in FIG. 1 comprises a scan gantry section 20, an operation unit 100, and a camera 120. The scan gantry section 20 and the camera 120 are disposed in an imaging room surrounded by a shielding material that blocks X-rays. In addition, the operation unit 100 is disposed in an operation room outside the imaging room.

The scan gantry section 20 comprises an X-ray source 22, a rotating plate 24, a collimator 26, an X-ray detector 28, a data collection unit 30, and a bed 32. The scan gantry section 20 comprises a rotating plate controller 50, a bed controller 52, an X-ray controller 54, and a high-voltage generation unit 56.

The X-ray source 22 irradiates the subject 1 placed on the bed 32 with X-rays. Examples of the X-ray source 22 include an X-ray tube device. The collimator 26 limits a range of irradiation with X-rays. The rotating plate 24 comprises an opening portion 25 into which the subject 1 placed on the bed 32 enters, is equipped with the X-ray source 22 and the X-ray detector 28, and rotates the X-ray source 22 and the X-ray detector 28 around the subject 1.

The X-ray detector 28 is disposed at a position facing the X-ray source 22. The X-ray detector 28 is a device that comprises a plurality of detection elements that detect X-rays transmitted through the subject 1 and that detects a spatial distribution of X-rays, and functions as a detection unit that detects a signal obtained from the subject 1. The detection elements of the X-ray detector 28 are arranged two-dimensionally in a rotation direction and a rotation axis direction of the rotating plate 24. The data collection unit 30 collects the spatial distribution of the X-rays detected by the X-ray detector 28 as digital data.

The rotating plate controller 50 controls the rotation and the tilt of the rotating plate 24. The bed controller 52 controls the forward and backward movement, widthwise movement, and elevation of the bed 32. The high-voltage generation unit 56 generates a high voltage to be applied to the X-ray source 22. The X-ray controller 54 controls the output of the high-voltage generation unit 56. The scan gantry section 20 is an example of a component of an imaging unit that images the subject of the present disclosure and that generates imaging data of the subject.

The operation unit 100 comprises an input device 102, an image generation unit 104, a display 106, a storage device 108, a system controller 110, and an automatic imaging position movement controller 111.

The input device 102 is used for inputting examination data such as a name, an examination date and time, and an imaging condition of the subject 1. The input device 102 may include a keyboard, a pointing device such as a mouse, and the like.

The image generation unit 104 generates a tomographic image using the digital data collected via the data collection unit 30. The image generation unit 104 is an example of a component of an image reconstruction unit that generates a reconstructed image based on the imaging data of the present disclosure.

The display 106 displays the tomographic image generated by the image generation unit 104, various types of information input by the input device 102, and the like. Examples of the display 106 include a liquid crystal display and an organic EL display. The input device 102 and the display 106 may be integrated by using a touch panel type display. It should be noted that EL is an abbreviation for electro-luminescence.

The storage device 108 stores the digital data collected by the data collection unit 30, the tomographic image generated by the image generation unit 104, a program executed by the system controller 110, data used by the program, and the like. The storage device 108 may be an HDD, an SSD, or the like. It should be noted that HDD is an abbreviation for Hard Disk Drive. SSD is an abbreviation for Solid State Drive.

The system controller 110 reads out programs corresponding to various functions of the medical image capturing system 10 from the storage device 108, and executes the various programs to realize various functions. That is, the system controller 110 controls various processing units such as the rotating plate controller 50, the bed controller 52, and the X-ray controller 54.

The system controller 110 acquires various types of input information transmitted from the input device 102, and transmits a control signal corresponding to the input information to each unit. The system controller 110 transmits, to the display 106, a display signal representing information to be displayed on the display 106.

The system controller 110 acquires a camera image of the subject 1 placed on the bed 32, which is transmitted from the camera 120. The system controller 110 transmits the camera image to the automatic imaging position movement controller 111.

The automatic imaging position movement controller 111 acquires the camera image, and specifies a position of an imaging part of the subject 1 based on the camera image. The position of the imaging part of the subject 1 may be represented by coordinate values in the camera image. The position of the imaging part of the subject 1 may be applied as coordinate values in a real space obtained by converting the coordinate values in the camera image. The camera image is an example of a captured image of the subject of the present disclosure.

The system controller 110 sets the imaging center in an imaging space of the scan gantry section 20. For example, the imaging center in the imaging space may be applied as coordinate values in a real space of a light localizer beam that is projected into the imaging space.

The automatic imaging position movement controller 111 performs automatic imaging position movement control of the bed 32 to move the bed 32 to a position where the imaging part of the subject 1 matches the imaging center of the imaging space, via the bed controller. The match may include aspects that have a deviation within a defined range. Details of the automatic imaging position movement controller 111 will be described below.

The camera 120 images the subject 1 placed on the bed 32 from above. The camera 120 may be disposed on a ceiling of the imaging room, or on top of the scan gantry section 20. In the present embodiment, a case where the camera 120 is disposed on the ceiling of the imaging room will be illustrated. A positional relationship of the camera 120 with the position of the bed 32 is adjusted. A mounting state of the camera 120 is adjusted during initial adjustment in start-up of the medical image capturing system 10. The adjustment of the mounting state of the camera 120 may be appropriately performed in a case where an abnormality occurs.

The camera image of the subject 1 placed on the bed 32, which is captured and acquired by using the camera 120, may be displayed on the display 106. An operator in the operation room can visually recognize the camera image of the subject 1 and can check a state of the subject 1. The camera image of the subject 1 may be used for movement control of the bed 32 to the imaging space for the medical image. The camera image of the subject 1 may be stored in the storage device 108.

The high-voltage generation unit 56 generates a tube voltage to be applied to the X-ray source 22 based on the imaging condition of the medical image set via the input device 102. The X-ray source 22 to which the tube voltage is applied irradiates the subject 1 with X-rays corresponding to the imaging condition.

The X-ray detector 28 detects the transmitted X-rays, which are X-rays emitted from the X-ray source 22 and transmitted through the subject 1, by using a plurality of detection elements, and acquires a spatial distribution of the transmitted X-rays.

A rotation operation of the rotating plate 24 is controlled by using the rotating plate controller 50. That is, the rotating plate 24 rotates based on the imaging condition acquired via the input device 102, particularly the setting of the rotation speed and the like.

The bed 32 is controlled by using the bed controller 52. That is, the bed 32 moves relative to the rotating plate 24, and moves the imaging part set on the subject 1 to an imaging field of view, which is a range in which the transmitted X-rays are detected.

While the rotating plate 24 is rotating, the irradiation with X-rays using the X-ray source 22 and the detection of X-rays using the X-ray detector 28 are repeated, and projection data, which is an X-ray projection image of the subject 1, is measured at various projection angles. The projection data is associated with a view representing each projection angle and a detection element number of the X-ray detector 28. The detection element number may include a channel number and a column number.

The measured projection data is transmitted to the image generation unit 104. The image generation unit 104 performs back projection processing on a plurality of projection data to generate a tomographic image. The generated tomographic image may be displayed on the display 106 as a medical image or may be stored in the storage device 108.

### Configuration Example of Automatic Imaging Position Movement Controller

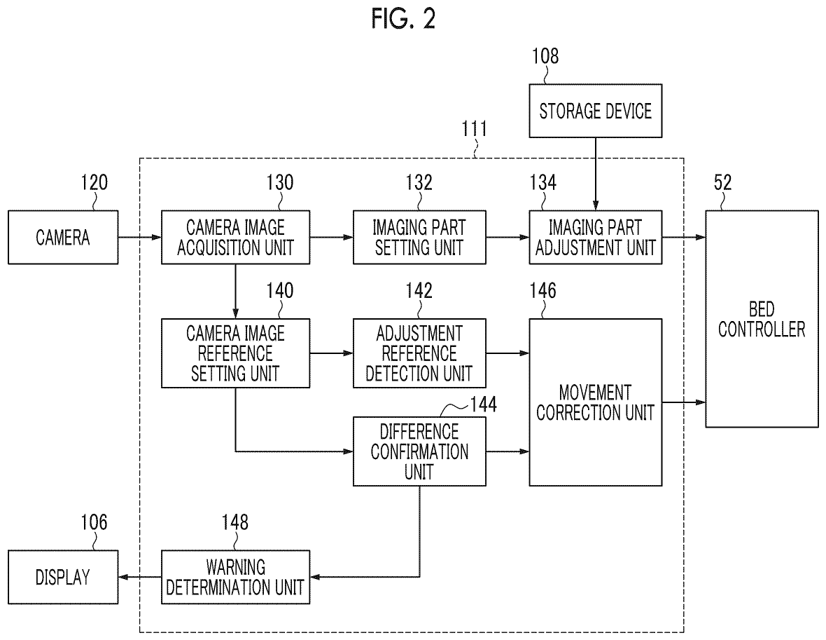

FIG. 2 is a functional block diagram showing an electric configuration example of an automatic imaging position movement controller. Prior to capturing a medical image for the subject 1, an imaging position of the subject 1 in capturing the medical image is automatically set using a camera image of the subject 1 placed on the bed 32. The bed controller 52 performs movement control of the bed 32 based on information on the imaging position of the subject 1 in capturing the medical image.

The automatic imaging position movement controller 111 comprises a camera image acquisition unit 130, an imaging part setting unit 132, and an imaging part adjustment unit 134. The storage device 108 stores adjustment data used for adjusting a position of the imaging part. The adjustment data may be generated for each facility and for each operator. For example, the adjustment data may be a table in which identification information for each facility is associated, or a table in which identification information for each operator is associated.

The camera image acquisition unit 130 acquires the camera image of the subject 1 acquired using the camera 120. The camera image may be a still image or a frame image constituting a video. The camera image acquisition unit 130 may acquire the camera image generated by the camera 120, or may acquire an imaging signal from the camera 120 and generate a camera image from the imaging signal. That is, the term “acquire” may include meanings such as generation of information and conversion of information.

The camera image acquisition unit 130 may acquire the camera image via the system controller 110 shown in FIG. 1, or may directly acquire the camera image from the camera 120 as shown in FIG. 2.

The imaging part setting unit 132 sets the imaging part of the subject 1 in capturing the medical image, based on the camera image acquired using the camera image acquisition unit 130. That is, the imaging part setting unit 132 sets the imaging part of the subject 1 in capturing the medical image by using a shape of the subject 1 extracted from the camera image, information on an examination site included in the imaging condition, and the like.

For example, in a case where the examination site is the chest, the imaging part is set based on a position of the chin and a position of the shoulders estimated from the shape of the subject 1 in the camera image. The imaging part setting unit 132 may include a trained model that has learned a shape of the human body and an imaging position of the medical image for each examination site.

The imaging part adjustment unit 134 adjusts the position of the imaging part set by using the imaging part setting unit 132. The adjustment data stored in the storage device 108 is used to adjust the position of the imaging part. The imaging part adjustment unit 134 may read out the adjustment data from the storage device 108 according to the identification information for each facility, the identification information for the operator, and the like.

The automatic imaging position movement controller 111 detects deterioration in the accuracy of calculation of movement parameters in the automatic imaging position movement caused by the misalignment of the mounting state of the camera 120, and corrects the movement control of the imaging part of the subject 1 to the imaging center of the imaging space in the scan gantry section 20 shown in FIG. 1.

The misalignment of the mounting state of the camera 120 means a deviation in the positional relationship between the camera 120 and the bed 32. The deviation in the positional relationship between the camera 120 and the bed 32 includes a deviation of the bed 32 in a width direction, a deviation of the bed 32 in a forward/backward direction, and a deviation in a rotation direction. Causes of the misalignment of the mounting state of the camera 120 include contact of the operator with the camera 120 and application of an external force to the camera 120 due to an earthquake.

The automatic imaging position movement controller 111 monitors the misalignment of the mounting state of the camera based on a difference between a camera image reference set in advance for the camera image and an adjustment reference shown in the camera image, and issues a warning according to a degree of the misalignment of the mounting state of the camera. In addition, the automatic imaging position movement controller 111 corrects the automatic imaging position movement according to the misalignment of the mounting state of the camera.

The automatic imaging position movement controller 111 is an example of a component of the medical image capturing support apparatus of the present disclosure. The camera image reference is an example of a captured image reference of the present disclosure.

The automatic imaging position movement controller 111 comprises a camera image reference setting unit 140, an adjustment reference detection unit 142, a difference confirmation unit 144, a movement correction unit 146, and a warning determination unit 148.

The camera image reference setting unit 140 sets the camera image reference. The camera image reference may be applied to a light localizer beam, an image marker, an edge of the bed 32, and the like in the camera image in a case where the camera 120 is properly disposed and properly adjusted. The camera image reference may represent a position in the camera image by using coordinate values applied to the camera image.

For example, the light localizer beam may be projected onto a position within an imaging field of view of the camera 120, and the light localizer beam in the camera image may be acquired as the camera image reference.

Here, a case where the camera 120 is properly disposed refers to a state in which a posture of the camera 120, such as a position and an orientation, is adjusted so as to fall within a defined error range with respect to a predefined posture.

The camera image reference may be physically attached to the bed 32, such as a seal and a tape attached to the bed 32, or may be optically displayed on the bed 32, such as rays emitted to the bed 32. The camera image in a case where the camera 120 is properly disposed and properly adjusted is an example of a second captured image of the present disclosure.

The adjustment reference detection unit 142 performs image processing, such as object detection, on the camera image to detect an adjustment reference included in the camera image. The adjustment reference is applied to the light localizer beam, the image marker, the edge of the bed 32, and the like included in the camera image. The adjustment reference may be physically attached to the bed 32, such as a seal and a tape attached to the bed 32, or may be optically displayed on the bed 32, such as rays emitted to the bed 32, as with the camera image reference.

For example, in a case where the light localizer beam projected onto the bed 32 is set as the camera image reference, the light localizer beam included in the camera image is detected as the adjustment reference. The position of the adjustment reference may be represented by using the coordinate values applied to the camera image. The camera image used for detecting the adjustment reference is an example of a first captured image of the present disclosure.

The difference confirmation unit 144 confirms a difference between the camera image reference and the adjustment reference. The confirmation of the difference may include an aspect of deriving an index representing the difference. The derivation may include calculation of an index value. The difference confirmation unit 144 determines whether or not the mounting state of the camera 120 is misaligned based on the difference between the camera image reference and the adjustment reference. That is, in a case where the difference between the camera image reference and the adjustment reference exceeds a first range, the difference confirmation unit 144 determines that the mounting state of the camera 120 is misaligned. On the other hand, in a case where the difference between the camera image reference and the adjustment reference is within the first range, the difference confirmation unit 144 may determine that the mounting state of the camera 120 is a normal state in which the mounting state is not misaligned. The first range may be defined based on an imaging condition of a medical image, such as an imaging part. The misalignment of the mounting state of the camera 120 is an example of the misalignment of the mounting state of the imaging apparatus of the present disclosure.

The difference confirmation unit 144 may determine whether or not the mounting state of the camera 120 is misaligned based on a difference in position between the camera image reference and the adjustment reference in a first direction and in a second direction orthogonal to the first direction. The orthogonality may include substantial orthogonality in which the directions intersect with each other with an error within a defined range with respect to the orthogonality.

The difference confirmation unit 144 may determine whether or not the mounting state of the camera 120 is misaligned based on a difference in the tilt with respect to the first direction or the second direction. For example, the first direction may be a forward direction or a backward direction of the bed 32 in the camera image, and the second direction may be a width direction of the bed 32 in the camera image.

The movement correction unit 146 performs correction calculation of the automatic imaging position movement based on the difference between the camera image reference and the adjustment reference. The movement correction unit 146 may include processing of converting a pixel position in the camera image into a position in the real space or processing of converting the number of pixels in the camera image into a distance in the real space in the correction calculation of the automatic imaging position movement.

The movement correction unit 146 transmits a result of the correction calculation of the automatic imaging position movement to the bed controller 52. The bed controller 52 corrects the automatic imaging position movement based on the result of the correction calculation of the automatic imaging position movement. The result of the correction calculation of the automatic imaging position movement is an example of a control signal representing the correction of the movement control of the bed based on the difference of the present disclosure.

The warning determination unit 148 determines whether or not the difference between the camera image reference and the adjustment reference is at a warning level. The warning determination unit 148 issues a warning in a case where the difference is at the warning level. FIG. 2 shows display of information representing a warning on the display 106 as an example of issuing the warning. The information representing the warning displayed on the display 106 may be text information. The text information may include a symbol, a figure, and the like. Sound information such as a warning sound or voice may be applied as the warning. The determination of whether or not the difference is at the warning level is an example of the determination of whether or not the difference of the present disclosure exceeds a second range.

### Hardware Configuration Example of Electric Configuration of Operation Unit

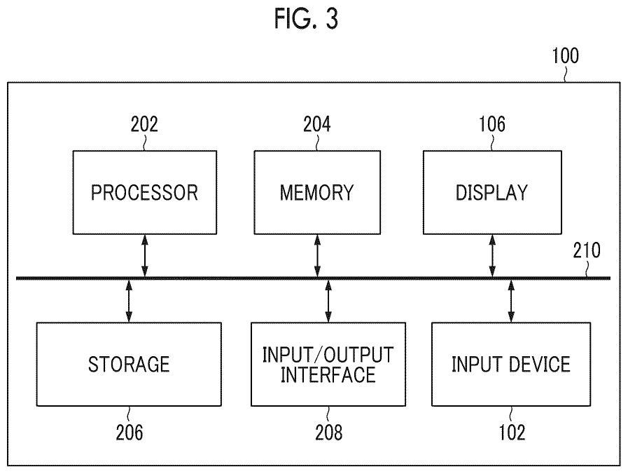

FIG. 3 is a block diagram showing a hardware configuration example of an electric configuration of an operation unit. Various processes of the operation unit 100 are realized by applying any computer. Any computer may have a processor that executes a program to execute various processes of the operation unit 100.

Any computer may be a general-purpose computer such as a personal computer or a computer for a specific use such as a server computer. Any computer may be a system such as a workstation or may be other hardware elements capable of executing a program, such as a virtual machine.

At least some of functions of the operation unit 100 may be realized using cloud computing. At least some of the functions of the operation unit 100 may be provided as SaaS. It should be noted that SaaS is an abbreviation for Software as a Service.

The operation unit 100 comprises a processor 202, a memory 204 as a main memory, a storage 206 as an auxiliary memory, an input/output interface 208, and a bus 210.

The processor 202 is connected to the memory 204, the storage 206, the input/output interface 208, the input device 102, and the display 106 via the bus 210.

The memory 204 includes a RAM. The memory 204 may include a ROM. The storage 206 may be, for example, a hard disk drive, a solid state drive, or a plurality of combinations thereof. In addition, the storage 206 may include an external storage device such as a removable medium.

It should be noted that RAM is an abbreviation for Random Access Memory, and ROM is an abbreviation for Read Only Memory. The hard disk drive may be referred to as HDD using an abbreviation for Hard Disk Drive. The solid state drive may be referred to as an SSD using an abbreviation for Solid State Drive.

A program, data, and the like for realizing various functions of the operation unit 100 are stored in the storage device including the memory 204 and the storage 206. The processor 202 executes the programs stored in the memory 204 to realize various functions. The processor 202 integrally controls each unit of the operation unit 100, various devices, units, and the like provided in the operation unit 100, and performs various processes.

The input/output interface 208 includes a communication interface that is connectable to an electric communication line such as a local area network, a connection interface that is connectable to an external device, and the like. As the connection interface that is connectable to an external device, for example, a universal serial bus or HDMI (HDMI is a registered trademark) can be applied. It should be noted that HDMI is an abbreviation for High-Definition Multimedia Interface.

The processor 202 communicates with various devices of the operation unit 100 via the input/output interface 208 to transmit and receive various types of information.

Examples of the input device 102 include a pointing device such as a keyboard and a mouse. The input device 102 may include a numeric keypad, various switch buttons, and the like. The input device 102 may include a voice input device. The input device 102 may be a touch panel type input device that is configured integrally with a display screen of the display 106.

The display 106 may be a liquid crystal display, an organic EL display, or a projector. The display 106 may be an appropriate combination of a liquid crystal display and the like. Various types of information are displayed on the display 106 in addition to the image captured by the operation unit 100. The display 106 is used as a part of the UI in a case of receiving an input via the input device 102. The present disclosure is not limited to one display 106, and a form of a multi-display comprising a plurality of display devices can be used. It should be noted that the organic EL may be referred to as OEL using an abbreviation for Organic Electro-Luminescence. UI is an abbreviation for User Interface.

In the present embodiment, each process is executed by any computer. In addition, any computer may be adapted to execute these processes using a processor, a program, or a combination of these. Any computer may be a general-purpose computer, a computer for a specific use, a system such as a workstation, or other hardware elements capable of executing a program.

One or a plurality of hardware may be applied to configure the processor 202, and the type of hardware is not limited. A programmable logic device such as a CPU, an MPU, and an FPGA may be applied as the hardware of the processor 202. A dedicated circuit that executes a specific process, such as an ASIC, may be applied as the processor 202. As the hardware of the processor 202, a GPU that performs a process specialized in image processing, an NPU that is specialized in AI processing, and the like may be applied.

The processor 202 functions as units, which are various processing units that execute various processes, and means, which are various processing means that execute various processes.

It should be noted that CPU is an abbreviation for Central Processing Unit, MPU is an abbreviation for Micro-Processing Unit, and FPGA is an abbreviation for Field-Programmable Gate Array. In addition, GPU is an abbreviation for Graphics Processing Unit, AI is an abbreviation for Artificial Intelligence, and NPU is an abbreviation for Neural network Processing Unit.

The processor 202 may be configured by combining different types of hardware. As the hardware of the processor 202, an electric circuit (circuitry) in which electric circuit elements such as semiconductor elements are combined is applied.

In a case where a plurality of pieces of hardware execute any one or more processes of the processor 202, the plurality of pieces of hardware may be located in devices physically separated from each other, or may be disposed in the same device. The order of the processes executed by the processor 202 is not limited to the order disclosed in the present specification and may be changed as appropriate. The hardware is configured by using an electric circuit or the like in which circuit elements such as semiconductor elements are combined.

Further, the present embodiment may be realized by applying hardware, software, firmware, microcode, or a combination thereof. The software, the firmware, and the microcode are configured by applying a program. For example, the program may be a program module group, and the functions of the software and the like may be realized by applying a processor that executes each function.

The program may be, for example, a program code stored in one or a plurality of non-transitory computer-readable media such as a storage medium and a storage, and a plurality of code segments. The program may be divided and stored in a plurality of non-transitory computer-readable media present in devices physically separated from each other.

The program code or the code segment may represent any combination of a procedure, a function, a subprogram, a routine, a subroutine, a module, a software package, a class, an instruction, a data structure, or a program statement. The program code or the code segment may be connected to another code segment or a hardware circuit by transmitting and receiving information, data, an argument, a parameter, or memory contents.

### Procedure of Automatic Imaging Position Movement Correction Method

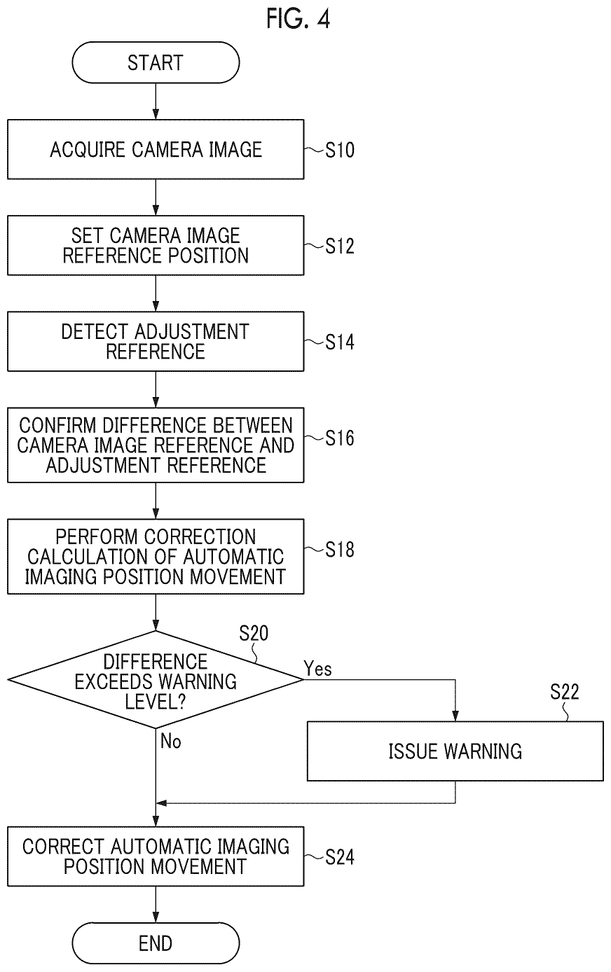

FIG. 4 is a flowchart showing a procedure of an automatic imaging position movement correction method. The automatic imaging position movement correction method of which the procedure is shown in FIG. 4 is realized by a computer functioning as the operation unit 100 shown in FIG. 1 executing a program.

In step S10, the camera image acquisition unit 130 acquires a camera image. The acquisition of the camera image may be executed in response to an operator's instruction input using the input device 102. In step S10, one camera image may be acquired, or a plurality of camera images may be acquired. The plurality of camera images may be a plurality of still images or a plurality of frame images included in a video.

In step S12, the camera image reference setting unit 140 sets a camera image reference. In step S12, the camera image reference stored in advance may be read out and set.

In step S14, the adjustment reference detection unit 142 detects an adjustment reference from the camera image acquired in step S10. In step S14, an adjustment reference selected from among candidates for a plurality of adjustment references may be selectively detected. In step S14, a position of the adjustment reference represented using coordinate values of the camera image may be derived.

In step S16, the difference confirmation unit 144 confirms a difference between the camera image reference and the adjustment reference, and determines whether or not the mounting state of the camera 120 is misaligned based on the difference. In step S16, it may be determined whether or not the mounting state of the camera 120 is misaligned based on a difference between the position of the camera image reference and the position of the adjustment reference.

In step S16, it is determined whether or not the difference between the camera image reference and the adjustment reference exceeds the first range, and, in a case where the difference between the camera image reference and the adjustment reference exceeds the first range, it is determined that the misalignment of the mounting state of the camera 120 has occurred.

In step S18, the movement correction unit 146 performs the correction calculation of the automatic imaging position movement. The movement correction unit 146 transmits a result of the correction calculation of the automatic imaging position movement to the bed controller 52.

In step S20, the warning determination unit 148 determines whether or not the difference between the camera image reference and the adjustment reference exceeds the warning level. In a case where it is determined that the difference exceeds the warning level, a determination result is “Yes”, and the process proceeds to step S22.

In step S22, the warning determination unit 148 issues a warning indicating that the difference between the camera image reference and the adjustment reference exceeds the warning level. The warning issued in step S22 may prompt maintenance such as adjustment of the camera 120. In a case where the warning is issued in step S22, the process proceeds to step S24.

On the other hand, in a case where it is determined in step S20 that the difference does not exceed the warning level, a determination result is “No”, and the process proceeds to step S24. In step S24, the movement correction unit 146 performs the correction of the automatic imaging position movement. That is, the movement correction unit 146 transmits the result of the correction calculation of the automatic imaging position movement calculated in step S18 to the bed controller 52. The bed controller 52 performs the corrected automatic imaging position movement control using the result of the correction calculation of the automatic imaging position movement.

The automatic imaging position movement correction method may be performed each time a medical image is captured, or may be performed in the start-up of the medical image capturing system 10 on each operating day. In a case where the medical image capturing system 10 is regularly maintained, the automatic imaging position movement correction method may be performed. In a case where an abnormality such as an emergency stop occurs in the medical image capturing system 10, the automatic imaging position movement correction method may be performed. In addition, the automatic imaging position movement correction method may be performed in response to an instruction from the operator. The automatic imaging position movement correction method is an example of an operation method of the medical image capturing support apparatus of the present disclosure.

### Specific Examples of Camera Image Reference and Adjustment Reference

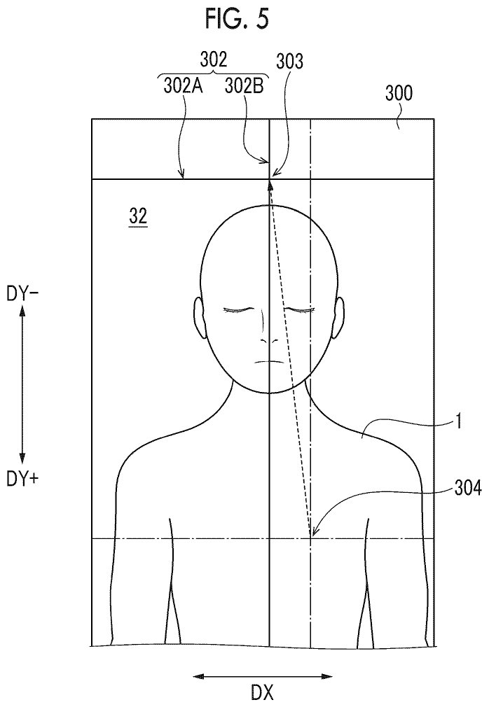

FIG. 5 is a schematic diagram of a camera image. FIG. 5 schematically shows a part of a camera image 300 in a case where the camera 120 shown in FIG. 1 is properly disposed and properly adjusted.

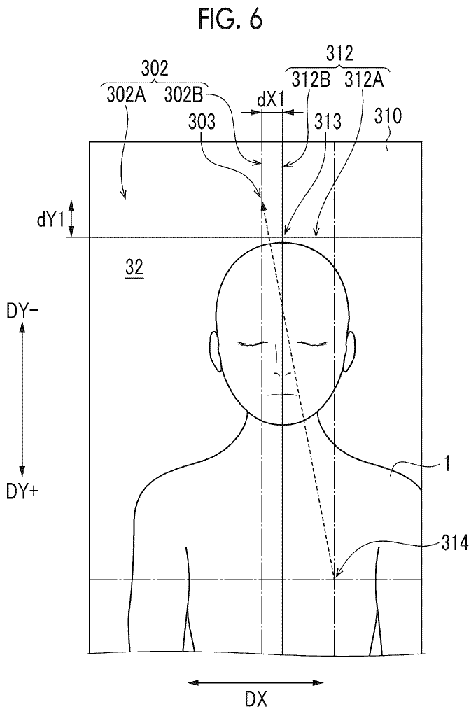

In FIG. 5, DY− represents a forward direction of the bed 32 toward the imaging space of the medical image, and DY+ represents a backward direction of the bed 32 away from the imaging space of the medical image. Hereinafter, in a case where there is no need to distinguish between the forward direction and the backward direction, both of the forward direction and the backward direction may be collectively referred to as a forward/backward direction. In addition, DX represents a width direction of the bed 32. The same applies to FIGS. 6 to 11.

The camera image 300 shown in FIG. 5 includes the subject 1 lying on his/her back in a head-first supine position. The camera image 300 schematically shows a light localizer beam 302 projected onto the bed 32 and an imaging part 304 of the subject 1 in capturing the medical image. In FIG. 5, the heart is illustrated as the imaging part 304.

The automatic imaging position movement is a function of moving the bed 32 such that the imaging part 304 of the subject 1 placed on the bed 32 moves to the imaging center defined in the imaging space of the medical image. The imaging center is understood as an intersection of the light localizer beams projected into the imaging space.

Two one-dot chain lines indicating the imaging part 304 shown in FIG. 5 are virtual lines that are not included in the actual camera image 300. In addition, a broken line with an arrow shown in FIG. 5 is a virtual line that schematically represents the automatic imaging position movement in a case where an intersection 303 of the light localizer beams 302 shown in FIG. 5 is assumed to be the imaging center of the imaging space of the medical image.

The imaging part setting unit 132 shown in FIG. 2 acquires a position of the imaging part 304 in the camera image 300 shown in FIG. 5. FIG. 5 illustrates a representative position of the heart, which is an examination site, as the position of the imaging part 304. The representative position of the examination site may be a geometric representative position of the examination site or may be an anatomical representative position of the examination site. Examples of the geometric representative position include a centroid.

The camera image reference setting unit 140 may set the light localizer beam 302 included in the camera image 300 as the camera image reference. FIG. 5 shows the light localizer beam 302 including a line segment 302A extending in the width direction of the bed 32 and a line segment 302B extending in the forward/backward direction of the bed 32.

The camera image reference setting unit 140 may set a position of the intersection 303 of the line segment 302A and the line segment 302B as the position of the camera image reference. The position of the intersection 303 in the camera image 300 may be represented by using the coordinate values applied to the camera image 300.

For example, the position of the camera image reference in the width direction of the bed 32 may be the coordinate values of the intersection 303 in the same direction. Similarly, the position of the camera image reference in the forward/backward direction of the bed 32 may be the coordinate values of the intersection 303 in the same direction.

The camera image reference setting unit 140 may set an image marker not shown in FIG. 5, an edge of the bed 32, or the like as the camera image reference. The camera image reference setting unit 140 may store the set camera image reference.

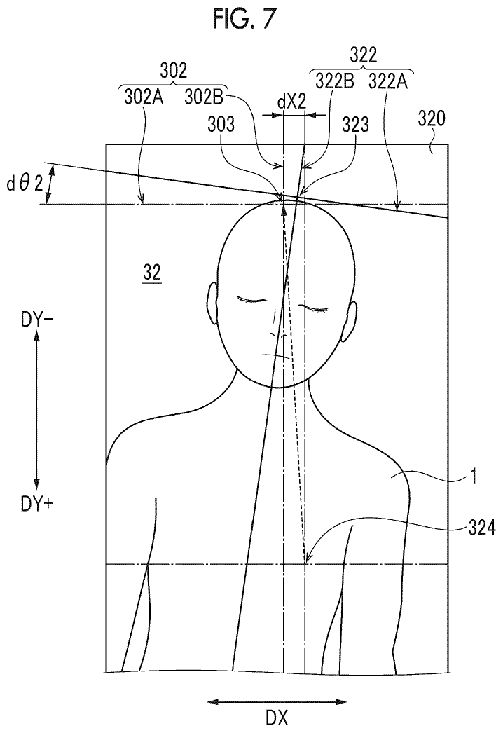

FIG. 6 is a schematic diagram of a camera image in a case where a mounting state of a camera is misaligned in front, rear, left, and right directions. In FIG. 6, the light localizer beams 302 shown in FIG. 5, which are not included in a camera image 310, are shown using one-dot chain lines. The same applies to FIG. 7.

Due to the misalignment of the camera 120 shown in FIG. 1 in the front, rear, left, and right directions, an imaging part 314 in the camera image 310 shown in FIG. 6 is shifted to the right in FIG. 6 in the width direction of the bed 32 and is shifted in the backward direction of the bed 32, relative to the imaging part 304 in the camera image 300 shown in FIG. 5.

Similarly, a light localizer beam 312 in the camera image 310 shown in FIG. 6 is shifted to the right in FIG. 6 in the width direction of the bed 32 and is shifted in the backward direction of the bed 32, relative to the light localizer beam 302 in the camera image 300 shown in FIG. 5. A shift amount in the width direction of the bed 32 in the camera image 310 shown in FIG. 6 is represented by a reference numeral dX1, and a shift amount of the bed 32 in the backward direction is represented by a reference numeral dY1.

The camera image reference setting unit 140 shown in FIG. 2 may set the light localizer beam 302 as the camera image reference in the camera image 310. The camera image reference setting unit 140 may set the coordinate values of the intersection 303 of the light localizer beams 302 as the position of the camera image reference in the camera image 310.

The adjustment reference detection unit 142 detects the light localizer beam 312 from the camera image 310 as the adjustment reference in the camera image 310. The adjustment reference detection unit 142 may acquire coordinate values of an intersection 313 of a line segment 312A extending in the width direction of the bed 32 and a line segment 312B extending in the forward/backward direction of the bed 32, which are included in the light localizer beam 312, as the position of the adjustment reference.

The difference confirmation unit 144 confirms the difference between the light localizer beam 302 and the light localizer beam 312 as the difference between the camera image reference and the adjustment reference. For example, as the difference between the camera image reference and the adjustment reference in the width direction of the bed 32, a difference between the coordinate values of the intersection 303 of the light localizer beams 302 and the coordinate values of the intersection 313 of the light localizer beams 312 may be calculated.

Specifically, the difference confirmation unit 144 may calculate a shift amount dX1 as the difference between the coordinate values of the intersection 303 of the light localizer beams 302 and the coordinate values of the intersection 313 of the light localizer beams 312 in the width direction of the bed 32, and may calculate a shift amount dY1 as the difference between the coordinate values of the intersection 303 of the light localizer beams 302 and the coordinate values of the intersection 313 of the light localizer beams 312 in the forward/backward direction of the bed 32.

The movement correction unit 146 performs the correction calculation of the automatic imaging position movement based on the difference between the light localizer beam 302 and the light localizer beam 312. For example, the movement correction unit 146 calculates a correction value of a moving distance of the automatic imaging position movement derived from the position of the imaging part 314 of the camera image 310 by using the shift amount dX1 in the width direction of the bed 32 and the shift amount dY1 in the forward/backward direction of the bed 32.

In the correction of the automatic imaging position movement based on the camera image 310 shown in FIG. 6, the automatic imaging position movement schematically shown in FIG. 6 using a broken line with an arrow is corrected to the automatic imaging position movement schematically shown in FIG. 5 using a broken line with an arrow.

FIG. 7 is a schematic diagram of a camera image in a case where the mounting state of the camera is rotationally misaligned. Due to the rotational misalignment of the mounting state of the camera 120 shown in FIG. 1, the subject 1 in a camera image 320 shown in FIG. 7 is rotated relative to the subject 1 in the camera image 300 shown in FIG. 5. FIG. 7 shows an example in which the subject 1 in the camera image 320 is shifted to the right in FIG. 7 in the width direction of the bed 32 and is rotated clockwise in FIG. 7.

Similarly, a light localizer beam 322 in the camera image 320 shown in FIG. 7 is shifted to the right in FIG. 7 in the width direction of the bed 32 relative to the light localizer beam 302 and is rotated clockwise in FIG. 7. The shift amount in the width direction of the bed 32 is represented by a reference numeral dX2, and the shift amount in the rotation direction is represented by a reference numeral dθ2. The shift amount in the rotation direction is synonymous with a rotation angle. A reference numeral 322A and a reference numeral 322B are line segments included in the light localizer beam 322 and represent line segments orthogonal to each other. In addition, a reference numeral 323 represents an intersection of the line segment 322A and the line segment 322B.

In a case where the light localizer beam 302 is set as the camera image reference and the light localizer beam 322 is detected as the adjustment reference, the difference confirmation unit 144 derives a difference between the light localizer beam 302 and the light localizer beam 322. Specifically, the difference confirmation unit 144 may calculate the shift amount dX2 in the width direction of the bed 32 and the shift amount dθ2 in the rotation direction.

The movement correction unit 146 performs the correction calculation of the automatic imaging position movement based on the difference between the light localizer beam 302 and the light localizer beam 322. For example, the movement correction unit 146 calculates a correction value of a moving distance of the automatic imaging position movement derived from the position of the imaging part 324 of the camera image 320 by using the shift amount dX2 in the width direction of the bed 32 and the shift amount dθ2 in the rotation direction.

In the correction of the automatic imaging position movement based on the camera image 320 shown in FIG. 7, the automatic imaging position movement schematically shown in FIG. 7 using a broken line with an arrow is corrected to the automatic imaging position movement schematically shown in FIG. 5 using a broken line with an arrow.

### Specific Example of Camera Image Reference

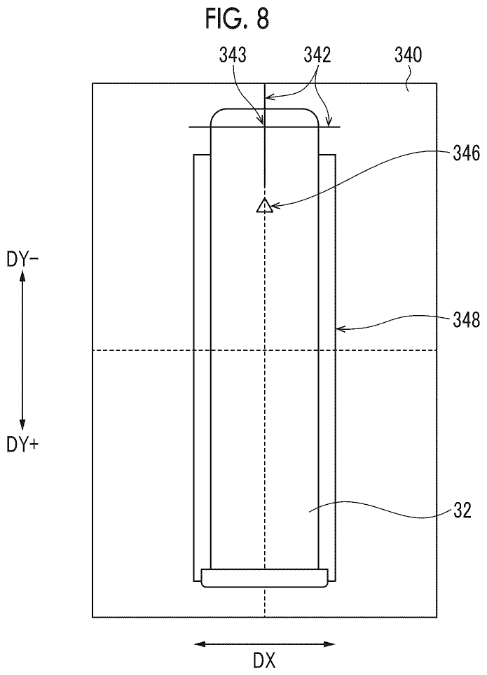

FIG. 8 is a schematic diagram of a camera image showing a specific example of the camera image reference. FIG. 8 shows a camera image 340 in which the entire bed 32 is included. Two broken lines orthogonal to each other, which are shown in FIG. 8, define the center of the camera image 340 and may be displayed on the camera image 340.

As the camera image reference, a light localizer beam 342 projected onto the bed 32 that has not entered the imaging space of the scan gantry section 20, an image marker 346, and a structure of the bed 32 such as an edge 348 of the bed 32 may be applied.

In the light localizer beam 342 shown in FIG. 8, a position of an intersection 343 in the width direction of the bed 32 matches a center position of the bed 32 in the same direction and matches a center position of the camera image 340 in the same direction. The position of the intersection 343 of the light localizer beams 342 in the forward/backward direction may be any position in the same direction, and is preferably a position separated from the imaging part 304 of the subject 1 by a defined distance.

In the image marker 346 shown in FIG. 8, a center position in the width direction of the bed 32 matches a center position of the bed 32 in the same direction and matches a center position of the camera image 340 in the same direction. The position of the image marker 346 in the forward/backward direction may be any position, and is preferably a position separated from the imaging part 304 of the subject 1 by a defined distance. FIG. 8 shows an example in which a centroid position of the image marker 346 is applied as the position of the image marker 346.

The edge of the bed 32 applied to the camera image reference may be an edge of the bed 32 on the right side in FIG. 8, may be an edge of the bed 32 on the left side in FIG. 8, may be a leading edge in the forward direction, or may be a trailing edge in the backward direction. The leading edge of the bed 32 in the forward direction is an upper edge in FIG. 8, and the trailing edge of the bed 32 in the backward direction is a lower edge in FIG. 8.

The camera image reference may be defined in the camera image 340 of the bed 32 on which the subject 1 is not placed, or may be defined in the camera image 300 in which the subject 1 is placed on the bed 32 as shown in FIG. 5.

### Specific Example of Adjustment Reference

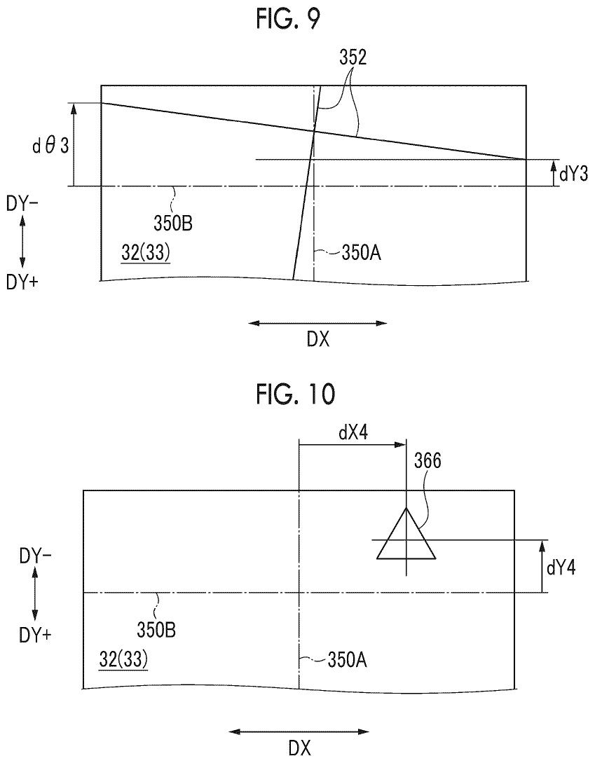

FIG. 9 is a schematic diagram showing a first specific example of the adjustment reference. FIG. 9 shows a part of a top plate 33 of the bed 32 on which the subject 1 is placed as shown in FIG. 5 and the like. In addition, in FIG. 9, a camera image reference 350A in the width direction of the bed 32 and a camera image reference 350B in the forward/backward direction of the bed 32 are shown using one-dot chain lines. The same applies to FIG. 10.

FIG. 9 illustrates a light localizer beam 352 projected onto the top plate 33 of the bed 32 as the first specific example of the adjustment reference. The light localizer beam 352 shown in FIG. 9 is shifted by a shift amount dY3 in the forward direction relative to the camera image reference 350B in the forward/backward direction of the bed 32, and is rotated by a shift amount dθ3 in the clockwise direction in FIG. 9.

FIG. 10 is a schematic diagram showing a second specific example of the adjustment reference. FIG. 10 illustrates an image marker 366 having a triangular shape as the second specific example of the adjustment reference. The image marker 366 is shifted by a shift amount dX4 to the right in FIG. 10 relative to the camera image reference 350A in the width direction of the bed 32.

In addition, the image marker 366 is shifted by a shift amount dY4 relative to the camera image reference 350B in the forward direction of the bed 32.

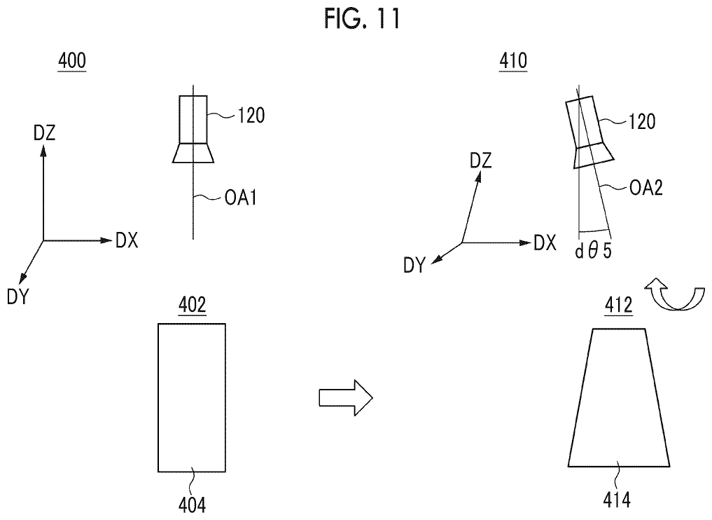

FIG. 11 is an explanatory diagram of a modification example of the image marker. A reference numeral DZ in FIG. 11 represents a vertically upward direction. A schematic diagram 400 shows an AR marker 404 in a camera image 402 captured by using the camera 120 that is properly attached and properly adjusted. The schematic diagram 400 shows the camera 120 that is properly attached and properly adjusted, with its imaging optical axis OA1 facing vertically downward.

A schematic diagram 410 shows an AR marker 414 in a camera image 412 captured by using the camera 120 with a misaligned mounting state. The schematic diagram 410 shows the camera 120 that is oriented so that its imaging optical axis OA2 faces a direction rotated by a rotational shift amount dθ5 relative to the vertical direction. It should be noted that AR is an abbreviation for Augmented Reality.

The AR marker 414 in the camera image 412 is visually recognized as a trapezoid due to the misalignment of the mounting state of the camera 120, rather than its original rectangular shape. The difference confirmation unit 144 shown in FIG. 2 may regard a difference in shape between the AR marker 404 that is visually recognized as a rectangle and the AR marker 414 that is visually recognized as a trapezoid, as the difference between the camera image reference and the adjustment reference.

The trapezoid referred to here may be a substantial trapezoid in which at least a part or all of the sides are not linear, for example, are curved, but can be recognized as a trapezoid as a whole.

The movement correction unit 146 may perform the correction calculation of the automatic imaging position movement based on the difference in shape between the AR marker 404 and the AR marker 414. The shape of the AR marker 404 is not limited to a rectangle, and need only be a shape that causes deformation in the camera image due to the misalignment of the mounting state of the camera 120.

### Effects of Embodiment

The medical image capturing system 10 and the automatic imaging position movement correction method according to the embodiment can obtain the following effects.
[1]

A camera image reference is set for the camera image 310 or the like acquired by imaging the subject 1 placed on the bed 32 using the camera 120 disposed on the ceiling or the like, and an adjustment reference is detected from the camera image 310 or the like. It is determined whether or not the mounting state of the camera 120 is misaligned, based on a difference between the camera image reference and the adjustment reference. As a result, the operator or the like can understand the misalignment of the mounting state of the camera 120.
[2]

Correction calculation of the automatic imaging position movement control of the bed 32 is performed based on the difference between the camera image reference and the adjustment reference. As a result, the automatic imaging position movement of the bed 32 based on a result of the correction calculation is corrected, and a reduction in accuracy of the automatic imaging position movement of the bed 32 is suppressed.
[3]

It is determined whether or not the difference between the camera image reference and the adjustment reference exceeds a warning level, and, in a case where the difference between the camera image reference and the adjustment reference exceeds the warning level, a warning is issued. As a result, the operator or the like is notified that the mounting state of the camera 120 is misaligned.
[4]

As the camera image reference and the adjustment reference, the light localizer beam projected onto the bed 32, the image marker, and the structure of the bed 32 are applied. As a result, the camera image reference can be set for the camera image 310 or the like, and the adjustment reference can be detected from the camera image 310 or the like.
[5]

A difference between a position of the camera image reference and a position of the adjustment reference is applied as the difference between the camera image reference and the adjustment reference. As a result, the misalignment of the mounting state of the camera 120 in the width direction of the bed 32 and in the forward/backward direction of the bed 32 can be determined.
[6]

A difference in the rotation direction in the camera image is applied as the difference between the camera image reference and the adjustment reference. As a result, the rotational misalignment of the mounting state of the camera 120 can be determined.
[7]

A difference between a shape of the camera image reference and a shape of the adjustment reference is applied as the difference between the camera image reference and the adjustment reference. As a result, it is determined whether or not the mounting state of the camera 120 is misaligned, based on a difference between the shape of the camera image reference and the shape of the adjustment reference.

### Application Example to Program and Program Product

The automatic imaging position movement correction method according to the embodiment may be configured as a program or a program product in which a processor or a computer including a processor realizes the functions of the respective steps.

For example, the program or the program product may cause the computer to realize a function of acquiring the camera image, a function of setting the camera image reference for the camera image, a function of detecting the adjustment reference from the camera image, and a function of determining whether or not the mounting state of the camera 120 is misaligned based on the difference between the adjustment reference and the camera image reference.

In addition, the program or the program product may cause the computer to realize a function of performing correction calculation of the automatic imaging position movement of the bed 32 based on the difference between the adjustment reference and the camera image reference, a function of correcting the automatic imaging position movement based on a result of the correction calculation, a function of determining whether or not the difference between the adjustment reference and the camera image reference is at a warning level, a function of issuing the warning, and the like.

The program or program product may be stored in a computer-readable medium that is a tangible non-transitory information storage medium, or may be provided through an information storage medium.

The present disclosure is not limited to the above-described embodiments, and various modifications can be made without departing from the spirit of the technical idea of the present disclosure. In addition, each of the first to fourth embodiments can be combined as appropriate.

### EXPLANATION OF REFERENCES

1: Subject
10: medical image capturing system
20: scan gantry section
22: X-ray source
24: rotating plate
25: opening portion
26: collimator
28: X-ray detector
30: data collection unit
32: bed
33: top plate
50: rotating plate controller
52: bed controller
54: X-ray controller
56: high-voltage generation unit
100: operation unit
102: input device
104: image generation unit
106: display
108: storage device
110: system controller
111: automatic imaging position movement controller
120: camera
130: camera image acquisition unit
132: imaging part setting unit
134: imaging part adjustment unit
140: camera image reference setting unit
142: adjustment reference detection unit
144: difference confirmation unit
146: movement correction unit
148: warning determination unit
202: processor
204: memory
206: storage
208: input/output interface
210: bus
300: camera image
302: light localizer beam
302A: line segment
302B: line segment
303: intersection
304: imaging part
310: camera image
312: light localizer beam
312A: line segment
312B: line segment
313: intersection
314: imaging part
320: camera image
322: light localizer beam
322A: line segment
322B: line segment
323: intersection
324: imaging part
340: camera image
342: light localizer beam
343: intersection
346: image marker
348: edge of bed
350A: camera image reference
350B: camera image reference
352: light localizer beam
366: image marker
400: schematic diagram
402: camera image
404: AR marker
410: schematic diagram
412: camera image
414: AR marker
dX1: shift amount
dX2: shift amount
dY1: shift amount
dY3: shift amount
dθ2: angle
dθ3: angle
dθ5: angle
OA1: imaging optical axis
OA2: imaging optical axis
S10 to S24: each step of automatic imaging position movement correction method

What is claimed is:

1. A medical image capturing support apparatus comprising:
a processor; and
a memory in which a program to be executed by the processor is stored,
wherein the processor is configured to
acquire a first captured image of a subject placed on a bed, the first captured image being acquired by imaging the subject using an imaging apparatus,
detect an adjustment reference defined on the bed from the first captured image,
derive a difference between a captured image reference set for the first captured image and the adjustment reference, and
determine misalignment of a mounting state of the imaging apparatus based on the difference.

2. The medical image capturing support apparatus according to claim 1,
wherein the processor is configured to determine that the mounting state of the imaging apparatus is misaligned in a case where the difference exceeds a first range.

3. The medical image capturing support apparatus according to claim 1,
wherein the processor is configured to output a control signal for correcting movement control of the bed based on the difference.

4. The medical image capturing support apparatus according to claim 1,
wherein the processor is configured to issue a warning in a case where the difference exceeds a second range.

5. The medical image capturing support apparatus according to claim 1,
wherein the processor is configured to detect a light localizer beam displayed on the bed as the adjustment reference.

6. The medical image capturing support apparatus according to claim 1,
wherein the processor is configured to detect an image marker represented on the bed as the adjustment reference.

7. The medical image capturing support apparatus according to claim 6,
wherein the processor is configured to detect a position of the image marker in the first captured image as a position of the adjustment reference.

8. The medical image capturing support apparatus according to claim 6,
wherein the processor is configured to
acquire a shape of the image marker represented on the bed as the captured image reference,
detect a shape of the image marker in the first captured image as the adjustment reference, and
derive a difference between the shape of the image marker represented on the bed and the shape of the image marker in the first captured image, as the difference.

9. The medical image capturing support apparatus according to claim 1,
wherein the processor is configured to detect a structure of the bed as the adjustment reference.

10. The medical image capturing support apparatus according to claim 1,
wherein the processor is configured to set the adjustment reference included in a second captured image acquired by using the imaging apparatus in a specified mounting state, as the captured image reference.

11. An operation method of a medical image capturing support apparatus, the operation method comprising causing a computer functioning as the medical image capturing support apparatus to execute:
acquiring a first captured image of a subject placed on a bed, the first captured image being acquired by imaging the subject using an imaging apparatus;
detecting an adjustment reference defined on the bed from the first captured image;
deriving a difference between a captured image reference set for the first captured image and the adjustment reference; and
determining misalignment of a mounting state of the imaging apparatus based on the difference.

12. A non-transitory, computer-readable tangible recording medium which records thereon, a program for causing a computer functioning as a medical image capturing support apparatus to realize:
a function of acquiring a first captured image of a subject placed on a bed, the first captured image being acquired by imaging the subject using an imaging apparatus;
a function of detecting an adjustment reference defined on the bed from the first captured image;
a function of deriving a difference between a captured image reference set for the first captured image and the adjustment reference; and
a function of determining misalignment of a mounting state of the imaging apparatus based on the difference.

13. A medical image capturing system comprising:
an imaging apparatus configured to image a subject and generate imaging data of the subject;
an image reconstruction unit configured to generate a reconstructed image based on the imaging data;
a processor; and
a memory in which a program to be executed by the processor is stored,
wherein the processor is configured to
acquire a first captured image of a subject placed on a bed, the first captured image being acquired by imaging the subject using an imaging apparatus,
detect an adjustment reference defined on the bed from the first captured image,
derive a difference between a captured image reference set for the first captured image and the adjustment reference, and
determine misalignment of a mounting state of the imaging apparatus based on the difference.
